(19) United States
(12) Patent Application Publication
(10) Pub. No.: US 2017/0049596 A1
(43) Pub. Date: Feb. 23, 2017

(54) IMPLANT DELIVERY SYSTEM AND METHOD OF USE

(71) Applicants: STRYKER CORPORATION, Kalamazoo, MI (US); STRYKER EUROPEAN HOLDINGS I, LLC, Kalamazoo, MI (US)

(72) Inventor: Jon Schabert, Dublin, CA (US)

(21) Appl. No.: 15/306,749

(22) PCT Filed: Apr. 27, 2015
(86) PCT No.: PCT/US15/27744

§ 371 Date: Oct. 25, 2016

Related U.S. Application Data
(60) Provisional application No. 61/986,718, filed on Apr. 30, 2014.

(51) Int. Cl. A61F 2/966; A61M 25/01

(52) U.S. Cl. CPC A61F 2/966; A61M 25/0108; A61F 2230/0069

(57) ABSTRACT

A tubular implant delivery system includes a delivery catheter having a distal marker and a proximal marker; and a tubular implant loaded in the delivery catheter, the tubular implant having a delivery length when in a collapsed, delivery configuration, and an implanted length shorter than the delivery length when in an expanded, implanted configuration, wherein a distance between the proximal marker and the distal marker on the delivery catheter is based upon a nominal implanted length of the tubular implant.

### RELATED APPLICATION DATA

The present application claims the benefit under 35 U.S.C. §119 to U.S. provisional patent application Ser. No. 61/986,718, filed Apr. 30, 2014. The foregoing application is hereby incorporated by reference into the present application in its entirety.

### FIELD OF THE INVENTION

The present disclosure pertains generally to systems and methods for delivering medical implants. More particularly, the present disclosure pertains to delivery systems and methods for delivering a tubular implant to a target site in a vasculature of a patient.

### BACKGROUND

The use of intravascular medical devices and implants has become an effective method for treating many types of vascular disease. In general, a suitable intravascular device is inserted into the vascular system of the patient and navigated through the vasculature to a target site in a patient. Using this method, virtually any target site in the patient's vascular system may be accessed, including the coronary, cerebral, and peripheral vasculature.

Catheters are often utilized to place medical implants, such as stents and embolic devices, at a desired location within a body. Usually, stents are tubular prosthesis for insertion through body lumens; although, stents may have a wide variety of sizes and shapes. A stent may be delivered by being mounted over a balloon and loaded onto a catheter, and after positioning the stent at the desired location, the balloon is inflated to expand the stent radially outward. Alternatively, a stent may be loaded onto a catheter in a reduced configuration and/or diameter; then introduced into the lumen of a body vessel. For example, self-expanding stents are to be delivered in an elastically compressed or collapsed state while being confined within a tubular restraining member, such as a catheter. The catheter is threaded through the vascular system until its distal end reaches the implantation site. Additionally, the catheter may be introduced into the patient over a guidewire which has been previously introduced, in the so-called “over-the-wire” and “rapid-exchange” delivery systems. The collapsed stent is typically mounted on a pusher member disposed within the catheter, so that the stent is introduced, advanced or pushed through the catheter. When the stent is positioned adjacent to the desired location, it is unsheathed by withdrawal of the catheter relative to the stent, and allowed to expand to a predetermined diameter in the body vessel, engaging the interior walls of the vessel, without requiring assistance from a balloon.

A self-expanding stent may be biased so as to expand upon release from the delivery catheter and/or includes a shape-memory component which allows the stent to expand upon exposure to a predetermined condition. Some stents may be characterized as hybrid stents which have some characteristics of both self-expandable and balloon expandable stents. In either configuration, once delivered to a target location within the body, the expanded stent supports and reinforces the vessel wall while maintaining the vessel in an open and unobstructed condition.

In some medical applications, such as bridging the neck of an aneurysm, diverting blood flow from an aneurysm or a blood vessel, an accurately implantation of a self-expanding stent or tubular implant in a target location is needed. Some implant delivery systems include a radio-opaque marker at the distal end tip of delivery catheters, so physicians may estimate a distal landing of the tubular implant when is pushed out of the catheter distal end tip having a marker, with the assistance of fluoroscopic imaging systems. However, when confined within a delivery catheter, a collapsed self-expanding stent or tubular implant usually has larger delivery length than its implanted length, when the stent or tubular implant is expanded in an implanted configuration, making it difficult for the physicians to estimate a proximal landing of the implanted stent or tubular implant. Thus, there may be an increased risk of failure and duration of the medical procedure, or at least, a failure to deliver the stent or tubular implant in a target location, to, for example, accurately bridge the neck of the aneurysm or divert the blood flow out of a blood vessel, as desired. Accordingly, there is an ongoing need to provide a delivery system for delivering self-expanding tubular implants that provides more accurate delivery and positioning of the implant at a target location.

### SUMMARY

In one embodiment of the disclosed inventions, a method of delivering a tubular implant to a target location in a body lumen is provided, wherein the tubular implant has a delivery length when in a collapsed, delivery configuration, and an implanted length shorter than the delivery length when in an expanded, implanted configuration. The method includes inserting an implant delivery system into the body lumen, until a distal marker located on a component of the implant delivery system is positioned adjacent a target distal landing location for a distal end of the tubular implant when implanted in the body lumen. A projected proximal landing location in the body lumen for a proximal end of the tubular implant when implanted in the body lumen is then identified based on a location of a proximal marker on a same or different component of the delivery system, while the distal marker remains positioned adjacent the target distal landing location, wherein a distance between the proximal marker and the distal marker corresponds to a nominal implanted length of the tubular implant

The nominal implanted length of the tubular implant may be based upon an actual implanted length of the tubular implant in a predetermined body lumen (e.g., a cerebral artery) having a standard cross-section. The act of identifying the projected proximal landing location may be further based upon an actual cross-section of the body lumen. The method may further include repositioning the delivery catheter if the identified projected proximal landing location is clinically undesirable. Optionally, the tubular implant may be biased to change from the delivery configuration to the implanted configuration when released out of the delivery catheter. Further, the tubular implant may be a blood flow diverter or a stent, and the body lumen may be a cerebral artery.

In various embodiments, the implant delivery system includes a delivery catheter through which the tubular implant is delivered to the body lumen, wherein one or both of the distal and proximal markers are located on the delivery catheter, and wherein the method further includes repositioning the delivery catheter prior to implantation of the tubular implant if the identified projected proximal landing location is clinically undesirable. The implant delivery system further comprising a pusher wire slidably disposed in the delivery catheter, wherein the tubular implant is mounted on the pusher wire for delivery out of an open end of the delivery catheter into the body lumen when the delivery catheter is withdrawn proximally relative to the delivery wire. For example, the proximal marker may be located on the pusher wire, while the distal marker is located on the delivery catheter.

In accordance with another embodiment of the disclosed inventions, a method is provided for delivering a tubular implant to a target location in a body lumen, the tubular implant having a delivery length when in a collapsed, delivery configuration, and an implanted length shorter than the delivery length when in an expanded, implanted configuration, the method including the steps or acts of (i) inserting a delivery catheter into the body lumen until a marker on a distal end of the delivery catheter is positioned adjacent a target distal landing location for a distal end of the tubular implant when implanted in the body lumen; and (ii) identifying a projected proximal landing location in the body lumen for a proximal end of the tubular implant when implanted in the body lumen based on a location of a proximal marker located on the delivery catheter, while the distal marker remains positioned adjacent the target distal landing location, wherein a distance between the proximal marker and the distal marker on the delivery catheter is based upon an actual implanted length of the tubular implant in a lumen having a standard cross-section. Identifying the projected proximal landing location may further be based upon an actual cross-section of the body lumen. The method may include the further step or act of repositioning the delivery catheter if the identified projected proximal landing location is clinically undesirable.

In accordance with yet another embodiment of the disclosed inventions, a tubular implant delivery system is provided, the system including a tubular implant having a delivery length when in a collapsed, delivery configuration, and an implanted length shorter than the delivery length when in an expanded, implanted configuration; a delivery catheter comprising a distal marker indicating a location of a distal end of the tubular implant when the tubular implant is in a ready-to-deploy position in the delivery catheter; a pusher wire slidably disposed in a delivery catheter, wherein the tubular implant is mounted on the pusher wire for delivery through and out an open end of the delivery catheter; and a proximal marker located on one of the delivery catheter and pusher wire, wherein a distance between the proximal marker and the distal marker on the delivery catheter when the tubular implant is in a ready-to-deploy position in the delivery catheter is based upon a nominal implanted length of the tubular implant. The nominal implanted length of the tubular implant is preferably an approximation of an actual implanted length of the tubular implant in a lumen having a standard cross-section.

In accordance with still another embodiment of the disclosed inventions, a tubular implant delivery system includes a tubular implant having a delivery length when in a collapsed, delivery configuration, and an implanted length shorter than the delivery length when in an expanded, implanted configuration; and a delivery catheter having a lumen sized for deploying the tubular implant there through, the delivery catheter comprising a distal marker indicating a location of a distal end of the tubular implant when the tubular implant is in a ready-to-deploy position in the delivery catheter, and a proximal marker located proximal of the distal marker, wherein a distance between the proximal marker and the distal marker on the delivery catheter is based upon a nominal implanted length of the tubular implant, wherein the nominal implanted length of the tubular implant is based upon an actual implanted length of the tubular implant in a predetermined body lumen having a standard cross-section.

In accordance with a still further embodiment of the disclosed inventions, a tubular implant delivery system includes a delivery catheter having a distal marker and a proximal marker; and a tubular implant (e.g., a stent or a blood flow diverter) loaded in the delivery catheter, the tubular implant having a delivery length when in a collapsed, delivery configuration, and an implanted length shorter than the delivery length when in an expanded, implanted configuration, wherein a distance between the proximal marker and the distal marker on the delivery catheter is based upon a nominal implanted length of the tubular implant, wherein the nominal implanted length of the tubular implant is based upon an actual implanted length of the tubular implant in a predetermined body lumen having a standard cross-section, and wherein the distal marker is overlying or otherwise indicating a location of a distal end of the tubular implant loaded in the delivery catheter.

Other and further aspects and features of embodiments of the disclosed inventions will become apparent from the ensuing detailed description in view of the accompanying figures.

### BRIEF DESCRIPTION OF THE DRAWINGS

FIG. 1 is a perspective view of a tubular implant delivery system constructed according to one embodiment of the disclosed inventions;

FIG. 2 is cross-sectional view of a method of delivering a tubular implant into a target site of a patient using the implant delivery system of FIG. 1.

FIGS. 3A-E are cross-sectional views of a method of delivering a tubular implant into a target site of a patient using the implant delivery system of FIG. 1; and

FIG. 4 is a perspective view of a tubular implant delivery system constructed according to another embodiment of the disclosed inventions.

FIG. 5 is a perspective view of a tubular implant delivery system constructed according to yet another embodiment of the disclosed inventions.

FIG. 6 is a perspective view of a tubular implant delivery system constructed according to still another embodiment of the disclosed inventions.

### DETAILED DESCRIPTION

For the following defined terms, these definitions shall apply, unless a different definition is set forth in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5). As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments of the disclosed inventions are described hereinafter with reference to the figures. The figures are not necessarily drawn to scale, the relative scale of select elements may have been exaggerated for clarity, and elements of similar structures or functions are represented by like reference numerals throughout the figures. It should also be understood that the figures are only intended to facilitate the description of the embodiments, and are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated.

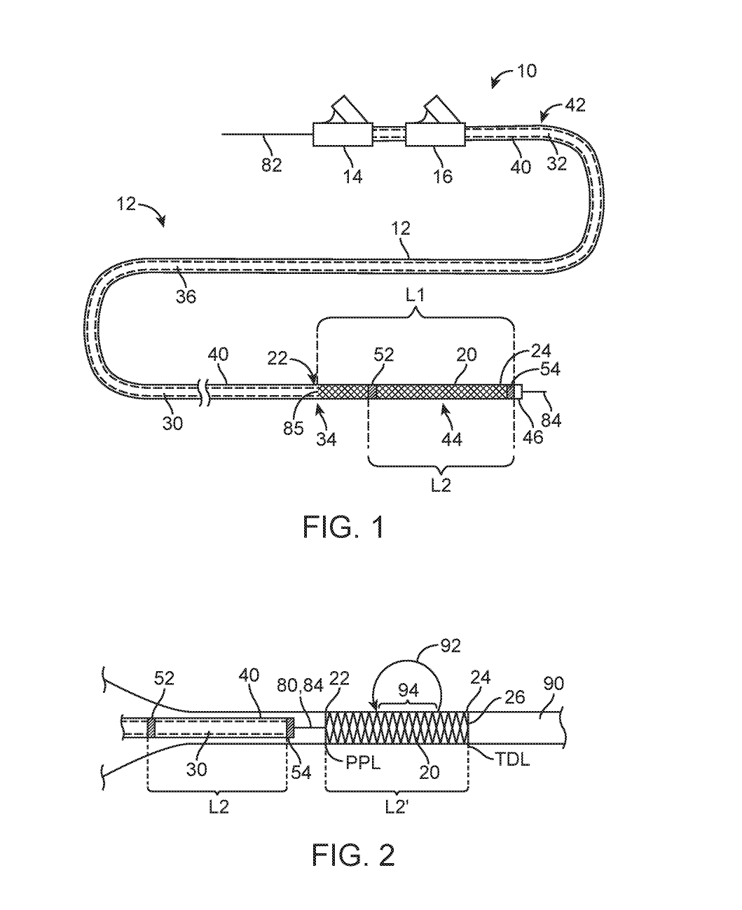

FIG. 1 is a perspective view of a medical assembly 10 for delivering an expandable tubular implant 20 into a target site of a patient, constructed in accordance with one embodiment of the disclosed inventions. The medical assembly 10 includes a tubular implant 20, such a stent or a flow diverter, and a delivery system 12 to which the tubular implant 20 is detachably coupled. The delivery system 12 and tubular implant 20 may be composed of suitable polymeric materials, metals and/or alloys, such as polyethylene, stainless steel or other suitable biocompatible materials or combinations thereof.

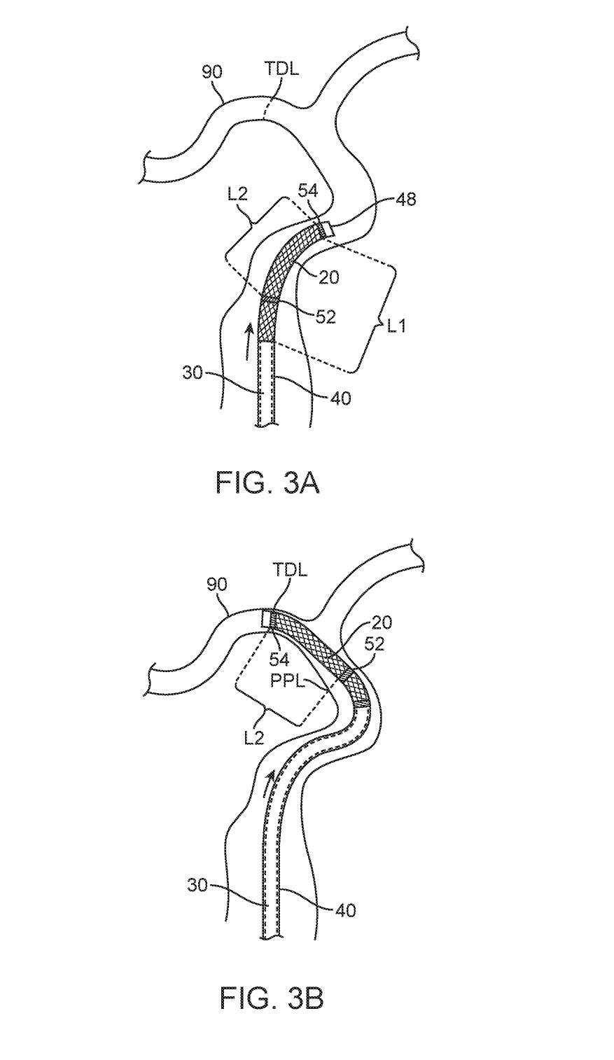

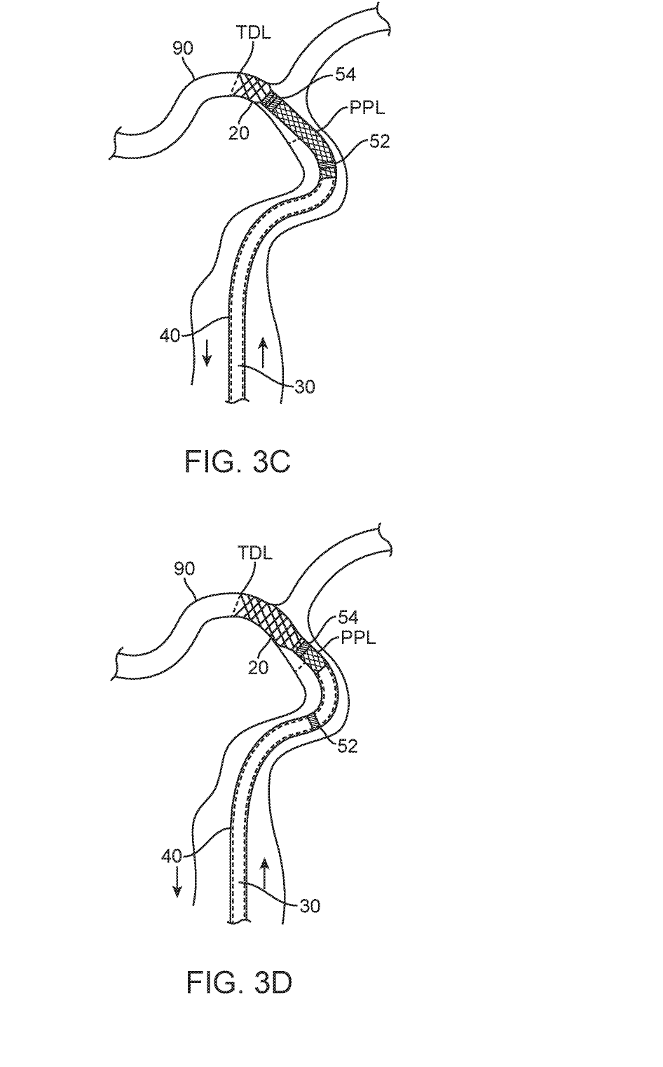

The delivery system 12 is dimensioned to reach remote locations of a vasculature and is configured to deliver the tubular implant 20 to a target location in a patient's body, such as an occlusion in a blood vessel, in a blood vessel adjacent to an aneurysm neck, a bifurcated blood vessel, or the like. The delivery system 12 includes a delivery configuration in which the tubular implant 20 is in a radially constrained and collapsed configuration, having a delivery length L1 (FIGS. 1, 3A-B). The delivery system 12 further includes a deployed configuration in which the tubular implant 20 is expand into a deployed configuration when deployed out of the delivery system 12, having an implanted length L2 (FIGS. 2, 3A) that is shorter than the delivery length L1, which will be described in more detail below.

The tubular implant 20 includes a tubular resilient member having a proximal end 22, a distal end 24, and defining an inner lumen 26 extending therebetween (FIG. 2). The tubular implant 20 is biased to extend radially outwards upon release from the delivery system 12. The tubular implant 20 may be constructed from a variety of materials such as stainless steel, elgiloy, nickel, titanium, nitinol, shape memory polymers, or combinations thereof. The tubular implant 20 may also be formed in a variety of manners as well. For example, the tubular implant 20 may be formed by etching or cutting a pattern from a tube or sheet of stent material; a sheet of stent material may be cut or etched according to a desired stent pattern whereupon the sheet may be rolled or otherwise formed into the desired substantially tubular, bifurcated or other shape. For the tubular implant 20, one or more wires or ribbons of stent material may be woven, braided or otherwise formed into a desired shape and pattern. The tubular implant 20 may include further components that are welded, bonded or otherwise engaged to one another. The tubular implant 20 may include a non-porous, non-permeable biocompatible material, cover or the like, when the tubular implant 20 is used as a blood flow diverter.

The delivery system 12 includes a delivery catheter 40 having a proximal end portion 42, a distal end portion 44 including an open distal end 46, and a delivery lumen 48 extending between the proximal end portion and the distal end portion 44. The delivery system further includes a pusher member 30 slidably disposed in the delivery lumen 48 of the delivery catheter 40. The pusher member 30 has a proximal end portion 32, a distal end portion 34, and a guidewire lumen 36 extending therebetween. The tubular implant 20 is disposed within the delivery catheter 40 and disposed distal of the pusher member 30, so that the pusher member 30 prevents proximal movement of the tubular implant 20 as the delivery catheter 40 is moved proximally for deployment of the tubular implant 20 out of the open distal end tip 46 of the delivery catheter 40, i.e., un-sheathing the tubular implant while the distal end of he implant remains at approximately the same location in the vessel) (FIG. 3B-D), as described in more detail below.

Alternatively or additionally, the distal end portion 34 of the pusher member 30 may comprise an actuator 85, including mechanical detachment interfaces, such as inflatable balloons, releasable interlocking geometries, mechanical fastening, or electrolytically actuated release mechanisms, or the like or combinations thereof, for deployment of the tubular implant 20 out of the distal end tip 46 of the delivery catheter 40 (not shown). When the actuator 85 includes a balloon, the balloon is in fluid communication with guidewire lumen 36 for inflation and deflation. An inflation source and/or vacuum (not shown) is fluidly coupled to the guidewire lumen 36 to deliver and withdraw fluid and/or gas to and from the balloon or the distal end tip 46 of the delivery catheter 40.

The tubular implant delivery system 12 includes side-arm adapters 14 and 16 in fluid communication with the delivery lumen 48 of delivery catheter 40 and the guidewire lumen 36 of the pusher member 30, respectively. The side-arm adapters 14 and 16 are configured to be coupled to syringes, fluid and/or vacuum sources (not shown). The delivery catheter 40 comprises a length about 50-300 cm, and typically about 60-200 cm. The delivery catheter 40 is configured for accessing a blood vessel or body lumen 90 for a desired treatment in a target site. For example, the target site may be within a small diameter blood vessel having a 2-5 mm lumen diameter and accessible by way of a tortuous vessel path which may involve sharp vessel turns and multiple vessel branches. In such cases, the delivery system 12, particularly the delivery catheter 40, has a small suitable diameter and flexible construction.

Further, the delivery system 12 includes a guidewire 80 having a proximal portion 82 and a distal portion 84. Generally, the proximal portion 82 may be formed from material that is stiffer than the distal portion 84 of the guidewire 80, so that the proximal portion has sufficient pushability to advance the guidewire 80 through the patient's vascular system, while the distal portion 84 may be formed of a more flexible material that remains flexible and tracks more easily to access remote locations in tortuous regions of the vasculature. In some instances, the proximal portion 82 of the guidewire 80 may include a reinforcement layer, such a braided layer or coiled layer to enhance the pushability of the guidewire 80. When using the delivery systems 12, the delivery catheter 40, the pusher member 30 and the implant 20 are introduced into the patient over the guidewire 80, which has been previously introduced. The guidewire 80 may extend through the entire length of the delivery catheter 40 and pusher member 30 through the lumen 36 (FIG. 1). Alternatively, the guidewire 80 may extend through only a distal portion of the delivery catheter 40 and pusher member 30, in the so called “rapid-exchange” delivery systems (not shown).

Referring back to delivery catheter 40, the distal end portion 44 of the delivery catheter 40 comprises a plurality of radiopaque markers, or at least, a proximal marker 52 and a distal marker 54 (FIGS. 1-3E). The radiopaque markers 52, 54 include biocompatible materials, such as platinum, gold, tungsten, or alloys thereof or other metals. The distal marker 54 on the delivery catheter 40 overlies or otherwise indicates a location of the distal end 24 of the tubular implant 20 when loaded in a ready-to-deploy position in the delivery system 12 (FIGS. 1, 3A-B).

As used in this specification, the term “ready-to-deploy position” refers to the location of the implant 20 within the catheter distal end portion 44 in close proximity to the open distal end tip 46 (as shown in FIG. 1), just prior to deployment of the implant 20 into the body lumen 90 by withdrawing the delivery catheter 40 relative to the implant 20, while the implant 20 is prevented from moving proximally by the pusher member 30. Thus, the distal end 24 of the implant 20, when in a “ready-to-deploy position” in the delivery catheter 40, is at a same or substantially same location as the distal end 24 of the implant 20 will be when deployed out of the delivery catheter 40. Additionally, the distal end 24 of the implant 20 may include a radiopaque marker (not shown) configure to assist with the overlying and location matching of the distal end 24 of the implant 24 with the distal marker 54 of the delivery catheter 40. The implant 20 can be placed in the “ready-to-deploy position” in the delivery catheter 40 before the delivery catheter 40, the pusher member 30 and the implant 20 are introduced into the patient over the guidewire 80 as a unit, as described in detail below. Alternatively, the delivery catheter 40 can be introduced into the patient before the implant 20 and the pusher member 30 are introduced into the proximal end portion 42 of the delivery catheter 40 and pushed to the distal end portion 44. In these latter embodiments, a radiopaque marker on the distal end 24 of the implant 20 facilitates in vivo alignment of the distal end 24 of the implant 20 with the distal end 46 (and distal marker 54) of the catheter 40.

The distal marker 54 is used to identify the distal landing location of the distal end 24 of the tubular implant 20 if the implant is implanted in the body lumen 90 at any point in time, by withdrawing the delivery catheter 40 relative to the implant 20 (FIGS. 2, 3B-C). Thus, a clinician may select a desired (or “target”) distal landing location by observing the body lumen 90 on an imaging device (e.g., a fluoroscope), and maneuvering the delivery catheter 40 until the distal marker 54 is positioned at the target distal landing location. The proximal marker 52 of the delivery catheter 40 is used to identify and/or determine a projected proximal landing location in the body lumen 90 for the proximal end 22 of the tubular implant 20, when implanted in the body lumen 90 with the distal end of the implant 20 at the target distal landing location (FIGS. 2, 3B-E).

In particular, the length between the proximal marker 52 and the distal marker 54 on the delivery catheter 40 is based upon a nominal implanted length L2 of the tubular implant 20 (FIGS. 1, 2). As used in this specification, the nominal implanted length L2 comprises an estimated length of the tubular implant 20 when in an expanded, implanted configuration. The nominal implanted length L2 is based on dimensions (e.g. length, diameter) of the tubular implant 20, and may also take in consideration, the “typical” dimensions (e.g., a “standard” cross-sectional diameter) of the particular body lumen. For example, a “standard” cross-section of a section of a cerebral artery that is a frequent location for a stenting procedure is about 4 mm. Thus, taking into consideration this standard cerebral artery cross-section of 4 mm and the dimensions of the tubular implant 20, a nominal implanted length is calculated to determine the length (or distance) between the proximal marker 52 and the distal marker 54 of the delivery catheter 40.

The tubular implant 20 is biased to expand from a constrained delivery configuration to an expanded implanted configuration when released out of the delivery catheter 40. When the tubular implant 20 is constrained within the catheter 40 in the radially constrained and collapsed configuration, the delivery length L1 (FIGS. 1, 3A-B) of the tubular implant 20 will normally be longer than the implanted length L2 (FIGS. 2, 3A). By providing a marker that locates the (or allows for accurately locating) the projected proximal landing location, the clinician can confirm whether the projected proximal landing location is clinically desirable, or at least satisfactory, prior to deploying the implant 20. In the event the projected proximal landing location is clinically undesirable, the clinician may reposition the delivery catheter 12 to a new location in which both the target distal landing location and the projected proximal landing location are clinically desirable or at least satisfactory locations prior to deploying the implant 20.

FIG. 2 illustrates the tubular implant 20 in an expanded, implanted configuration using the delivery system 12 of FIG. 1. After gaining access to the vasculature region of a patient, the delivery catheter 40 and the pusher member 30 are introduced into the patient over the guidewire 80, which has been previously introduced into the vessel. The delivery catheter 40 is advanced until the distal marker 54 is located adjacent a target distal landing location for the implant beyond the neck 94 of an aneurysm 92. The clinician then determines the location of the projected proximal landing location for the implant based on the location of the proximal marker 52, while maintaining the position of the distal marker at the target distal landing location. If the projected proximal landing location is deemed suitable, and the implant will bridge the aneurysm neck 94, the tubular implant 20 is then deployed. As shown in FIG. 2, the actual implanted length L2′ of the tubular implant 20 in its expanded, implanted configuration is about the same as the distance between the proximal marker 52 and distal marker 54 on the delivery catheter 40 (i.e., its nominal implanted length L2).

FIGS. 3A-E illustrate an exemplary method of delivering the tubular implant 20 to a target site in a body lumen 90 using the delivery system 12. After gaining access to the vasculature region of a patient, the delivery catheter 40 having the tubular implant 20 loaded therein and disposed within the delivery lumen 46, is inserted into the body lumen 90 (FIG. 3A). The delivery catheter 40 is inserted until the distal marker 54 on the catheter 40 overlies or otherwise indicates that the location of the distal end 24 of the tubular implant 20 is positioned adjacent a target distal landing location for the distal end 24 of the tubular implant 20 when implanted in the body lumen 90 (FIG. 3B). The projected proximal landing location in the body lumen 90 is then determined for the proximal end 22 of the tubular implant 20 based on the location of a proximal marker 52 on the delivery catheter 40, while the distal marker 54 remains positioned adjacent the target distal landing location (FIG. 3B). As with the prior embodiments, the distance between the proximal marker 52 and the distal marker 54 on the delivery catheter 40 is based upon a nominal implanted length L2 of the tubular implant 20. The clinician then verifies that the proximal landing location is suitable for implantation of the implant 20, or otherwise repositions the delivery catheter 40, until both the target distal landing location and the projected proximal landing location are at suitable locations in the body lumen 90. The tubular implant 20 is then advanced out the open distal end 46 of the delivery catheter 40 and into the body lumen 90 at the target site by withdrawing the delivery catheter 40 relative to the pusher member 30, so that the distal end of the implant 20 stays in substantially the same location as the targeted distal landing location (FIG. 3C-D). After the tubular implant 20 is delivered and implanted at the target site (FIG. 3E), the delivery system 12 is withdrawn from the body lumen 90 of the patient (FIG. 3E).

In accordance with embodiments of this method, the nominal implanted length L2 of the tubular implant 20 may be based upon an actual implanted length L2′ of the tubular implant 20 in a body lumen 90 having a standard cross-section (e.g. 4 mm). Additionally, identifying the projected proximal landing location may be further based upon an actual cross-section of the body lumen 90. The clinician may determine the cross-section of the body lumen 90 (e.g. standard, non-standard or actual) with the assistance of an imaging system, such as a fluoroscope, or the like. The determination of the cross-section of the body lumen 90 may be performed prior to the medical procedure, to assist with the identification of the delivery system 12, distance between markers 52, 54, and/or the dimensions of the tubular implant 20 to be used in the medical procedure.

It is appreciated that the nominal implanted length L2 of the implant 20 is based on the “typical” dimensions of a body lumen and that the actual implanted length L2′ will vary depending on the actual dimensions of the body lumen 90 into which the implant 20 is delivered. Accordingly, the clinician may determine the actual diameter of the body lumen 90 either prior to or during the medical procedure to further refine the determination of the proximal landing location of the implant 20 in the body lumen 90.

As described above, the tubular implant 20 may be biased to change from the delivery configuration to the implanted configuration when released out of the delivery catheter 40. The tubular implant 20 for use in this method may comprise a blood flow diverter or stent. Further, the body lumen 90 may be a cerebral artery.

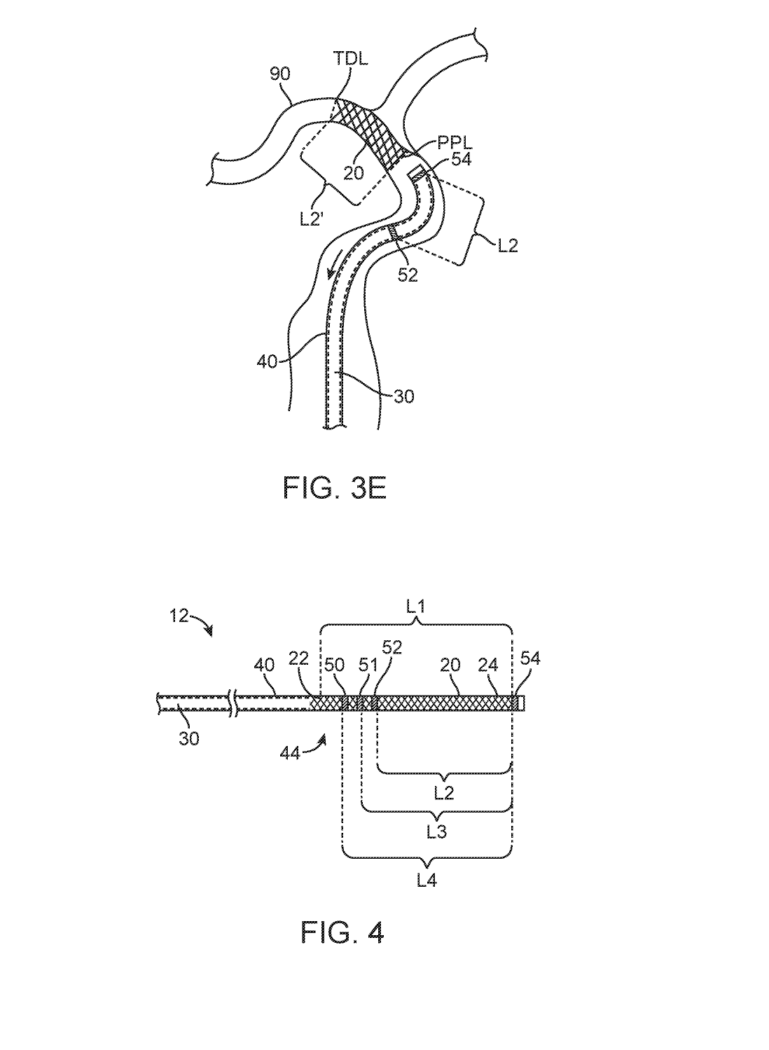

FIG. 4 is a perspective view of a medical system 12 distal end portion for delivering an expandable tubular implant 20 into a target site of a patient, constructed in accordance with another embodiment of the disclosed inventions. The distal end portion 44 of the delivery catheter 40 comprises a plurality of radiopaque markers, for example, a first proximal marker 50, a second proximal marker 51 and a third proximal marker 52 and a distal marker 54. The distal marker 54 on the catheter 40 overlies or otherwise indicates a location of the distal end 24 of the tubular implant 20 when loaded in a ready-to-deploy position in the delivery system 12. The proximal markers 50, 51 and 52 of the catheter 40 are configured to identify and/or determine a projected proximal landing location in the body lumen 90 for the proximal end 22 of the tubular implant 20 when implanted in the body lumen 90, depending on the dimensions of the tubular implant 20 that is used and/or the cross-section of the body lumen 90, in which the implant 20 is to be deployed and implanted.

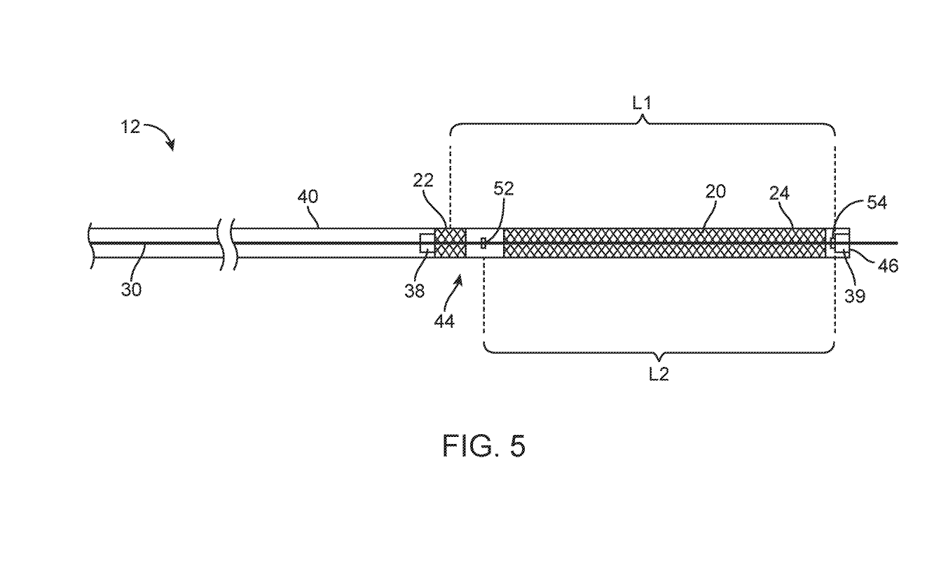

It will be appreciated that the exemplary method illustrated in FIGS. 3A-E may also be practiced using the delivery systems 12 of FIGS. 4. Those skilled in the art will appreciate that the delivery systems 12 and methods described herein may be contemplated to deliver tubular prosthesis, implants, stents, fluid diverters or the like for vascular and non-vascular application. In another embodiment illustrated in FIG. 5, the proximal and distal markers 52, 54 are located on the distal portion 84 of the pusher member 30 instead of on the catheter 40.

In FIG. 5, portions of the tubular implant 20 overlying the proximal and distal markers 52, 54 on the pusher member 30 are shown in shadow to facilitate visualization of the markers 52, 54. In this embodiment, the pusher member 30 is a pusher wire 30 having proximal and distal bumpers 38, 39 disposed thereon and configured to constrain axial movement of a tubular implant 20 disposes on the pusher wire 30 and between the proximal and distal bumpers 38, 39. The radiopaque markers may include the biocompatible materials described above. The distal marker 54 on the pusher wire 30 overlies or otherwise indicates a location of the distal end 24 of the tubular implant 20, which will also be the implanted location, i.e., the distal landing location, when the tubular implant 20 is implanted in body lumen. As used in conjunction with this embodiment, the term “ready-to-deploy position” refers to the location of the implant 20 on the pusher wire 30, between the proximal and distal bumpers 38, 39 and within the catheter distal end portion 44 in close proximity to open distal end tip 46, just prior to deployment of the implant 20 into the body lumen by withdrawing the delivery catheter relative to the implant 20.

Thus, the distal end 24 of the implant 20, when in a “ready-to-deploy position” in the delivery catheter 40, is at a same or substantially same location as the distal end of 24 the implant 20 will be when deployed out of the catheter 40. The distal marker 54 is configured to identify and/or determine a target distal landing location of the distal end 24 of the tubular implant 20 in the body lumen 90, as described above. The proximal marker 52 of the guidewire 80 is configured to identify and/or determine a projected proximal landing location in the body lumen 90 for the proximal end 22 of the tubular implant 20, as described above. The distal end 46 of the catheter includes a radiopaque marker (not shown) used to align the distal end 24 of the implant 20 with the distal end 46 of the catheter 40, to thereby facilitate delivery of the distal end 24 of the implant 20 to a target distal landing location, as described above. Additionally, the distal end 46 of the catheter 40 may also include a radiopaque marker (not shown) configure to assist with alignment of the distal end 34 of the implant 20 and the distal end 46 of the catheter 40.

Because the proximal and distal markers 52, 54 are located on the pusher wire 30 instead of the catheter 40 in this embodiment, this delivery system 12 is particularly suited for a delivery method wherein the catheter 40 to be advanced to the target location in the body lumen 90 before the tubular implant 20 and the pusher wire 30 are advanced through the catheter 40 to the target location.

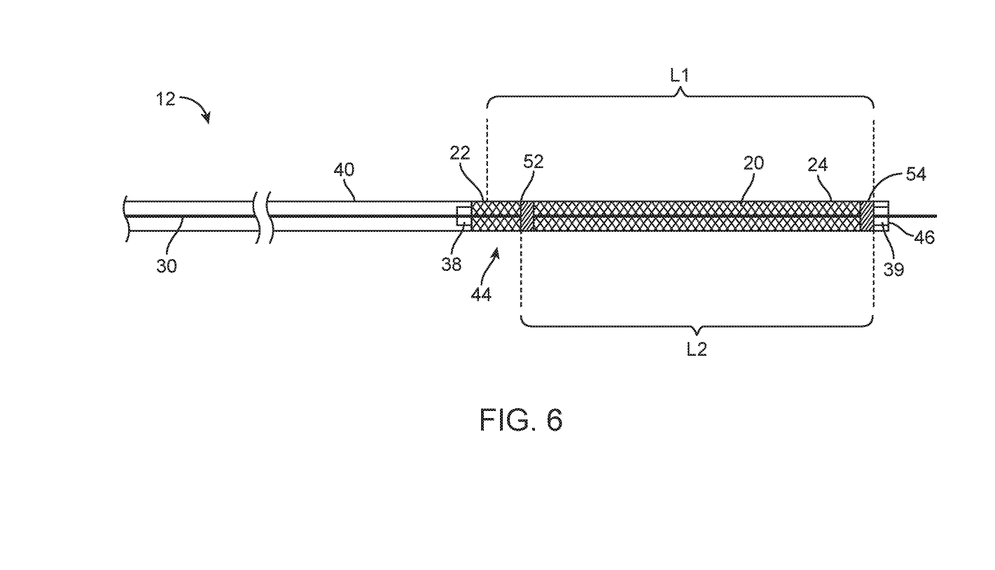

In still another embodiment depicted in FIG. 6, the proximal and distal markers 52, 54 are located on the tubular implant 20 instead of on the pusher wire 30 or the catheter 40. The distal ends of the catheter 40 and the pusher wire 30 may each include a radiopaque marker (not shown) configured to assist with positioning the distal end 24 of the tubular implant 20 at the target distal landing location, as described above. While the proximal and distal markers 52, 54 in the above-described embodiments are located on the same part of the medical assembly 10, the markers 52, 54 can be located on different parts thereof. For instance, the proximal marker 52 may be located on the pusher wire 30 or the tubular implant 20, while the distal marker 54 is located on the catheter 40.

Although particular embodiments have been shown and described herein, it will be understood that they are not intended to limit the disclosed inventions, and it will be apparent that various changes and modifications may be made (e.g., to the dimensions of various parts) without departing from the scope of the disclosed inventions, which are to be defined only by the claims and their equivalents. For instance, it will be appreciated that elements or components shown with any embodiment herein may be used on or in combination with other embodiments disclosed herein. The specification and drawings are, accordingly, to be regarded in

1. A method for delivering a tubular implant to a target location in a body lumen, the tubular implant having a delivery length when in a collapsed, delivery configuration, and an implanted length shorter than the delivery length when in an expanded, implanted configuration, the method comprising:
inserting an implant delivery system into the body lumen, until a distal marker located on a component of the implant delivery system is positioned adjacent a target distal landing location for a distal end of the tubular implant when implanted in the body lumen; and
identifying a projected proximal landing location in the body lumen for a proximal end of the tubular implant when implanted in the body lumen based on a location of a proximal marker on a same or different component of the delivery system, while the distal marker remains positioned adjacent the target distal landing location,
wherein a distance between the proximal marker and the distal marker corresponds to a nominal implanted length of the tubular implant.

2. The method of claim 1, wherein the nominal implanted length of the tubular implant is an approximation of an actual implanted length of the tubular implant in a lumen having a standard cross-section.

3. The method of claim 1, wherein identifying the projected proximal landing location is further based upon an actual cross-section of the body lumen.

4. The method of claim 1, the implant delivery system comprising a delivery catheter through which the tubular implant is delivered to the body lumen, wherein one or both of the distal and proximal markers are located on the delivery catheter.

5. The method of claim 4, further comprising repositioning the delivery catheter prior to implantation of the tubular implant if the identified projected proximal landing location is clinically undesirable.

6. The method of claim 5, the implant delivery system further comprising a pusher wire slidably disposed in the delivery catheter, wherein the tubular implant is mounted on the pusher wire for delivery out of an open end of the delivery catheter into the body lumen when the delivery catheter is withdrawn proximally relative to the delivery wire.

7. The method of claim 6, wherein proximal marker is located on the pusher wire and the distal marker is located on the delivery catheter.

8. The method of claim 1, the tubular implant comprising a stent or a blood flow diverter.

9. A method for delivering a tubular implant to a target location in a body lumen, the tubular implant having a delivery length when in a collapsed, delivery configuration, and an implanted length shorter than the delivery length when in an expanded, implanted configuration, the method comprising:
inserting a delivery catheter into the body lumen until a marker on a distal end of the delivery catheter is positioned adjacent a target distal landing location for a distal end of the tubular implant when implanted in the body lumen; and
identifying a projected proximal landing location in the body lumen for a proximal end of the tubular implant when implanted in the body lumen based on a location of a proximal marker located on the delivery catheter, while the distal marker remains positioned adjacent the target distal landing location,
wherein a distance between the proximal marker and the distal marker on the delivery catheter is based upon an actual implanted length of the tubular implant in a lumen having a standard cross-section.

10. The method of claim 9, wherein identifying the projected proximal landing location is further based upon an actual cross-section of the body lumen.

11. The method of claim 9, further comprising repositioning the delivery catheter if the identified projected proximal landing location is clinically undesirable.

12. The method of claim 9, wherein the method is performed using a tubular implant delivery system, the tubular implant delivery system comprising
a tubular implant having a delivery length when in a collapsed, delivery configuration, and an implanted length shorter than the delivery length when in an expanded, implanted configuration;
a delivery catheter comprising a distal marker indicating a location of a distal end of the tubular implant when the tubular implant is in a ready-to-deploy position in the delivery catheter;
a pusher wire slidably disposed in a delivery catheter, wherein the tubular implant is mounted on the pusher wire for delivery through and out an open end of the delivery catheter; and
a proximal marker located on one of the delivery catheter and pusher wire, wherein a distance between the proximal marker and the distal marker on the delivery catheter when the tubular implant is in a ready-to-deploy position in the delivery catheter is based upon a nominal implanted length of the tubular implant.

13. The method of claim 12, wherein the nominal implanted length of the tubular implant is an approximation of an actual implanted length of the tubular implant in a lumen having a standard cross-section.

14-17. (canceled)

18. The method of claim 12, the tubular implant comprising a stent or a blood flow diverter.
